(19) United States
(12) Patent Application Publication
(10) Pub. No.: US 2025/0001159 A1
(43) Pub. Date: Jan. 2, 2025

(54) SYSTEMS AND METHOD FOR IMPROVING CARDIORENAL SYNDROME

(71) Applicant: ABIOMED, Inc., Danvers, MA (US)

(72) Inventors: Michael Cuchiara, Danvers, MA (US); Soo Young Kim, Danvers, MA (US); Sathya Dev Unudurthi, Danvers, MA (US); Ralf Westenfeld, Danvers, MA (US)

(73) Assignee: ABIOMED, Inc., Danvers, MA (US)

(21) Appl. No.: 18/758,409

(22) Filed: Jun. 28, 2024

Related U.S. Application Data
(60) Provisional application No. 63/523,893, filed on Jun. 28, 2023.
(60) Provisional application No. 63/604,024, filed on Nov. 29, 2023.

(51) Int. Cl. A61M 60/33; A61M 60/13; A61M 60/139; A61M 60/152; A61M 60/17; A61M 60/178; A61M 60/216; A61M 60/515; A61M 60/894; A61M 60/90

(52) U.S. Cl. CPC A61M 60/33; A61M 60/13; A61M 60/139; A61M 60/152; A61M 60/17; A61M 60/178; A61M 60/216; A61M 60/515; A61M 60/894; A61M 60/90; A61M 2205/04; A61M 2205/103; A61M 2205/3327; A61M 2205/3331; A61M 2205/50; A61M 2210/125; A61M 2210/127

(57) ABSTRACT

Systems and techniques for improving cardiorenal syndrome (CRS) may be provided. The systems may include a first flow enhancer configured to increase a renal artery pressure. The systems may include a second flow enhancer or flow restrictor configured to reduce a renal vein pressure. Each flow enhancer or flow restrictor may be configured to increase a transrenal pressure gradient, improve filtering and renal perfusion.

### CROSS-REFERENCE TO RELATED APPLICATIONS

The present application claims priority to U.S. Provisional Patent Application Nos. 63/523,893, filed 28 Jun. 2023, and 63/604,024, filed 29 Nov. 2023, the contents of which are each incorporated by reference herein in its entirety.

### TECHNICAL FIELD

The present application is drawn to systems for treating or preventing cardiorenal syndrome, such as to systems of flow modifiers (such as flow enhancers or restrictors) configured to spare renal injury and improve renal function by, e.g., increasing a transrenal pressure gradient, and/or improving renal perfusion.

### BACKGROUND

Cardiorenal syndrome (CRS) encompasses a spectrum of acute or chronic disorders of heart and kidney function characterized by mutual deterioration, although it can be classified by which organ initiates CRS. For example, acute or chronic disfunction in one organ can induce acute or chronic disfunction in the other organ. In one example, cardiac initiated CRS may include a condition in which acute heart failure or acute coronary syndrome leads to a decline in renal function, such as in Glomerular Filtration Rate (GFR) (i.e., Type I CRS). In another example, chronic heart failure may result in chronic kidney disease (i.e., Type II CRS). An example of renal initiated CRS could be situations acute renocardiac syndrome, where acute kidney injury leads to CRS (i.e., Type III CRS) or chronic renocardiac syndrome where chronic kidney disease leads to chronic HF (i.e., Type IV CRS). Finally, CRS can occur secondary to non-renal or non-cardiac initiators such as amyloidosis, sepsis, cirrhosis (i.e., Type V CRS).

### BRIEF SUMMARY

In various aspects, a system for improving renal function and sparing renal injury may be provided. The system may include flow modifiers, not limited to flow enhancers or restrictors (which includes both flow reducers and blockers), where the flow modifiers may each, independently, be configured to increase a renal artery pressure, increase renal blood flow, reduce renal vein pressure, and/or increase renal blood flow, etc. The system may include a first flow modifier, which in some embodiments may be a flow enhancer configured to increase a renal artery pressure and renal blood flow. The system may include a second flow modifier, which may be a flow enhancer or restrictor configured to reduce a renal vein pressure and increase renal blood flow.

Each flow modifier may be configured to increase a transrenal pressure gradient and/or improve renal perfusion, thereby sparing renal injury and improving renal function (including GFR/filtering, diuresis, natriuresis, etc.).

The first flow modifier may be a passive, non-powered, wing or nozzle. The first flow modifier may be a microaxial pump. The microaxial pump may have an inlet in a superior portion of an inferior vena cava (IVC) or right atrium (RA), and an outlet in a pulmonary artery. The microaxial pump may have an inlet in an inferior vena cava (IVC) superior to a renal vein, and an outlet in the inferior vena cava (IVC) or right atrium (RA). The distance between microaxial pump inlet and outlet (e.g., cannula length) may be adjusted to (1) control where the inlet and outlet fall in the vascular and cardiac system (2) modulate recirculation between inlet and outlet, (3) modulate resistance to flow through the cannula. An artificial valve (such as a balloon or stent valve) may be disposed between the inlet and the outlet.

The first flow modifier may be operably coupled to a controller. The controller may be configured to receive information from a sensor. The controller may be configured to, based on the information, determine when a heart is in diastole. The controller may be configured to increase flow generated by the first flow modifier during diastole.

The second flow modifier may be a passive, non-powered, wing or nozzle. The second flow modifier may be a microaxial pump. The microaxial pump may have an inlet in a left ventricle (LV) and an outlet in an aorta. The microaxial pump may have an inlet in a descending aorta and an outlet superior to a renal artery. An artificial valve (such as a balloon or stent valve) may be disposed between the inlet and the outlet. The flow restrictor may be a balloon.

In various aspects, a method for improving renal function and sparing renal injury may be provided. The method may include increasing a transrenal pressure gradient by increasing a renal artery pressure, decreasing a renal vein pressure, or a combination thereof to increase renal perfusion, spare renal injury, and improve renal function.

The method may include providing a first flow modifier, as disclosed herein, configured to increase a renal artery pressure. The method may include inserting the first flow modifier such that an inlet may be disposed in a superior portion of an inferior vena cava (IVC) or right atrium (RA), and an outlet may be disposed in a pulmonary artery. The method may include inserting the first flow modifier such that an inlet may be disposed in an inferior vena cava (IVC) superior to a renal vein, and an outlet may be disposed in the inferior vena cava (IVC) or right atrium (RA).

The method may include providing a second flow modifier, as disclosed herein, configured to reduce a renal vein pressure. The second flow modifier may be a microaxial pump. The method may include inserting the microaxial pump such that an inlet may be disposed in a left ventricle (LV) and an outlet may be disposed in an aorta. The method may include inserting the microaxial pump such that an inlet may be disposed in a descending aorta and an outlet may be disposed superior to a renal artery.

The method may include receiving information related to when a heart is in diastole. The method may include increasing flow generated by the first flow modifier during diastole.

The method may include determining an adjustment to a renal artery pressure or a renal vein pressure based on information from one or more sensors. The information from one or more sensors may be related to renal function, renal injury, volume status changes, a biomarker for heart failure status, or hemodynamics. The information related to renal function may include information related to urine output, serum creatine, GFR, BUN/creatine ratio, natriuresis (e.g. serum and or urine sodium) or a combination thereof. The information related to renal injury may include neutrophil gelatinase-associated lipocalin (NGAL), or Cystatin C, NAG, TIMP-2, IGFBP-7, KIM 1, or a marker of renal oxygenation. The information related to volume status changes may include information related to urine output, edema, signs and symptoms of congestion, or a combination thereof. The biomarker for heart failure status may be a natriuretic peptide.

The method may include determining an adjustment to the first flow modifier based on information from a sensor on the second flow modifier. The method may include determining an adjustment to the second flow modifier based on information from a sensor on the first flow enhancer.

The method may include, after achieving a desired transrenal pressure gradient, controlling the transrenal pressure gradient by adjusting a renal artery pressure, a renal vein pressure, or a combination thereof to increase renal perfusion, spare renal injury, and improve renal function. In various aspects, a method may be provided for determining treatment of a patient with signs and symptoms of congestion, or with markers of end organ function not in a normal range. The method may include recommending a first flow modifier (such as a flow enhancer) configured to increase a renal artery pressure if the patient is experiencing hypoperfusion. The method may include recommending a second flow modifier (such as a flow restrictor) configured to reduce a renal vein pressure if the patient is experiencing venous congestion.

The method may include determining a patient is experiencing hypoperfusion if a systolic blood pressure <90 mmHg, a cardiac index <2.2 L/min/m², the patient has cold extremities, the patient has elevated lactate or other indicators of renal hypoperfusion such as renal injury markers (e.g., NGAL, or Cystatin C, TIMP-2, NAG, IGFBP-7, KIM 1, or a marker of renal oxygenation). The method may include determining a patient is experiencing venous congestion if central venous pressure (CVP) or right atrial pressure (RAP)>10 mmHg, the patient has jugular vein distention, high intraabdominal pressure or the patient has peripheral edema.

The method may include providing a first flow modifier, as disclosed herein, configured to decrease a renal vein pressure. The method may include inserting the first flow modifier such that an inlet may be disposed in a superior portion of an inferior vena cava (IVC) or right atrium (RA), and an outlet may be disposed in a pulmonary artery. The method may include inserting the first flow modifier such that an inlet may be disposed in an inferior vena cava (IVC) superior to a renal vein, and an outlet may be disposed in the inferior vena cava (IVC) or right atrium (RA).

The method may include providing a second flow modifier, as disclosed herein, configured to increase a renal artery pressure. The method may include inserting the second flow modifier such that an inlet may be disposed in a left ventricle (LV) and an outlet may be disposed in an aorta. The method may include inserting the second flow modifier such that an inlet may be disposed in a descending aorta and an outlet may be disposed superior to a renal artery.

In various aspects, a method for cardiorenal syndrome (CRS) therapy may be provided. The method may include disposing a therapy-delivery device within a patient, where the therapy-delivery device is a blood pump. The method may include receiving information related to cardiorenal function. The method may include adjusting a therapy delivered by the therapy-delivery device based on the information related to renal function. The method may include, before disposing the therapy-delivery device within the patient, determining an initial cardiorenal function of the patient. The method may include, before disposing the therapy-delivery device within the patient, determining if a pusher or puller therapy is indicated, based on the initial cardiorenal function.

In certain aspects, the therapy-delivery device may include a blood pump disposed in puller therapy configuration. In certain aspects, the therapy-delivery device may include a blood pump disposed in a pusher therapy configuration. The information related to cardiorenal function may be a hemodynamic parameter, a renal function parameter, or a combination thereof. The hemodynamic parameter is renal blood flow, systolic blood pressure, cardiac index, or a combination thereof. The renal function parameter may be an estimated glomerular filtration rate (eGFR), a direct glomerular filtration rate (GFR), a urinary output, a urinary sodium concentration, a total urinary sodium content, or a renal injury marker. The renal injury marker may be Neutrophil gelatinase-associated lipocalin (NGAL) or Cystatin C. The cardiorenal function may be a measure of hypoperfusion and/or venous congestion.

In certain aspects, receiving information may include receiving data from a user. In certain aspects, receiving information may include receiving information includes receiving data from a real-time diagnostic sensor. The real-time diagnostic sensor may be integrated into the therapy-delivery device. The real-time diagnostic sensor may be a separate device from the therapy-delivery device, such as an implantable device or a wearable device. The real-time diagnostic sensor may be configured to detect an optical change in response to a measured biomarker. The real-time diagnostic sensor may be configured to detect a change in electrical properties in response to a measured biomarker. The real-time diagnostic sensor may be configured to detect a mechanical change in response to a measured biomarker.

Adjusting the therapy may include increasing or decreasing therapy dose. Increasing or decreasing the therapy dose may include increasing or decreasing a rotational speed of a blood pump. Adjusting the therapy may include increasing or decreasing an intended duration of therapy. Adjusting the therapy may include increasing or decreasing a duty cycle of therapy. The method may include determining adequacy of therapy withdrawal timing and duration.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1-3 are illustrations of various configurations of “pusher” therapies.

FIGS. 4-6 are illustrations of various configurations of “puller” therapies.

FIGS. 7-8 are flowcharts of various treatments using “pusher” and/or “puller” therapies.

FIG. 9A is a block diagram of an embodiment of a system.

FIG. 9B is a block diagram of an alternate embodiment of a system.

FIGS. 10A, 10B, and 10C are illustrations of embodiments of a system.

FIG. 11 is a flowchart of an embodiment of a method for controlling a therapy-delivery device.

FIG. 12 is a flowchart of a method.

FIGS. 13 and 14 are flowcharts of alternative embodiments of methods for controlling a therapy-delivery device based on a parameter related to renal function.

FIG. 15 is an schematic illustration of a blood pump assembly.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, may be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

As is known, cardiorenal syndrome (CRS) encompasses a spectrum of acute or chronic disorders of heart and kidney function characterized by mutual deterioration. For example, acute or chronic disfunction of one of the organs can induce acute or chronic disfunction in the other. Current therapies for cardiorenal syndrome are limited to devices and/or pharmacologic agents configured to change the fluid status in the body (e.g., diuretics or volume loading or ultrafiltration or dialysis) or to change hemodynamic status (e.g., inotropes, pressors, extracorporeal membrane oxygenation). These technologies do not specifically address the fundamental lack of flow into the kidney or out of the kidney without also affecting other organ systems with off-target effects. As described herein, the inventors have recognized that new treatment options responsive to CRS are desirable, such as new methods and devices for achieving hemodynamic effects.

In some instances, for a first order hemodynamic effect, the difference between renal artery pressure and renal vein pressure, which represents the transrenal pressure gradient, may be managed for driving blood filtration across the glomerulus (e.g., glomerular filtration rate, GFR) and/or for improving renal function (e.g., natriuresis, diuresis, congestion relief, etc.) and sparing of renal injury. Improvements in GFR, which is the gold standard marker for renal function in some instances, may be associated with reduced incidences of acute kidney injury (AKI), worsening renal function (WRF), and improved diuresis in patients with congestion. Therapies that seek to either increase renal artery pressure, such as by pushing more blood in the renal artery and increasing blood flow into the kidneys, (referred to herein as a “Pusher” therapies or pumps) or decrease renal vein pressure, such as by pulling more blood flow out of the renal vein, (referred to herein as “Puller” therapies), or both, may have the potential to increase the transrenal pressure gradient and, thus increase GFR and/or improve renal perfusion. In some instances, this may improve patient outcomes.

### Pusher Therapies

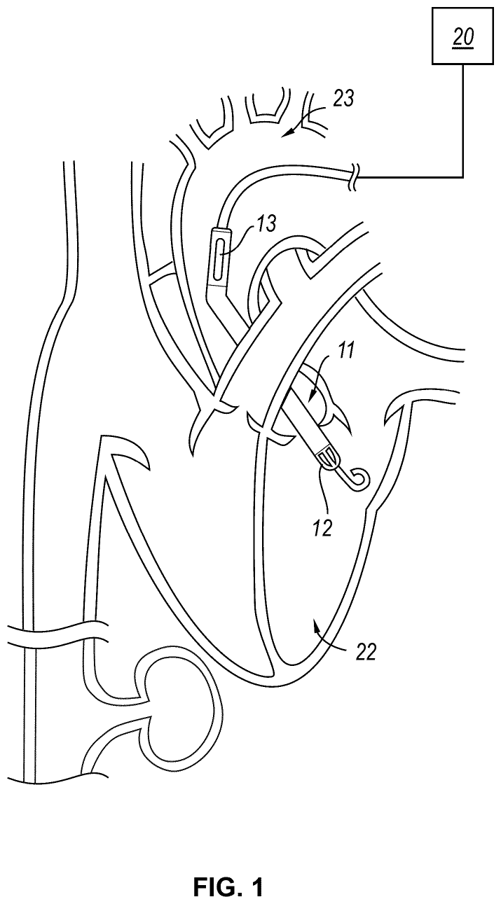

As described herein, various anatomical locations and configurations can provide a Pusher therapy to the patient. A first configuration, shown in FIG. 1, may include a microaxial flow pump (11), such as a percutaneous blood pump, which includes an inlet (12) that can be positioned within the left ventricle (LV) (22) of a patient and an outlet (13) that can be positioned in the aorta (23) of the patient. Such a configuration may augment diastole.

All such flow pumps disclosed herein may have various structures. For example, in some embodiments, blood may be configured to flow from the inlet, past a rotor or impeller, and out an outlet. A cannula may optionally be disposed upstream or downstream from rotor or impeller.

The location of the flow pump may have the potential to improve blood flow and pressure to the kidney. As will be understood, whether a flow pump is in a venous or arterial pump location, it may have the potential to improve the transrenal pressure gradient, improve renal perfusion, spare renal injury, and/or improve renal function. However, the most direct effect of the pump may vary based on location. For example, for pumps placed in the venous system, the most direct effect may be to decrease renal vein pressure. For pumps placed in the arterial system, the most direct effect may be to increase renal artery pressure. Both configurations may have the potential to improve the transrenal pressure gradient.

Placing the pump inlet within the LV may also have the added effect of unloading the LV and, and thus, reducing its hemodynamic and metabolic workload.

When a pump is disposed across the valve in the heart (in either the pusher or puller configuration) it may unload the heart and impact the kidney to some degree. When the pump inlet is placed in the aorta (e.g., in a pusher configuration) it may unload the heart less than when the inlet is within the LV but may more directly impact the kidney. When the pump is placed in the IVC (e.g., in a puller configuration) it may unload the kidney but not the right ventricle (RV). If the RV is healthy, such an approach may be acceptable. However, if the RV is not healthy (e.g., the RV is poor), this may lead to RV failure and the LV may fail to fill. Thus, matching pump location and configuration to patient type should be include an assessment or understanding of the impact of such location and configuration on the heart.

Further, this configuration (with the inlet within the LV) may stimulate the aortic and carotid baroreceptors to decrease overall sympathetic tone, including renal sympathetic tone. In some embodiment, this may improve renal function. As will be appreciated, in this location, the outlet of the pump may be positioned far from the renal artery and renal artery pressure increases may be modest.

In some embodiments, as shown in FIG. 1, the pump may be operable coupled to a controller 20. As will be appreciated, such a controller may be configured to increase and/or decrease a level of support provided by the pump (e.g., the level of flow). In some embodiments, the level of support may be adjusted to achieve a desired Pusher therapy of the pump.

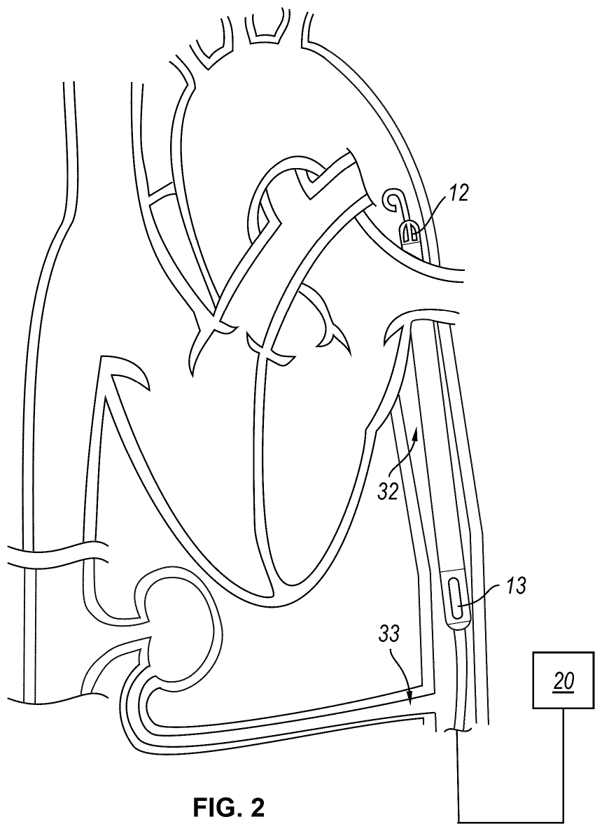

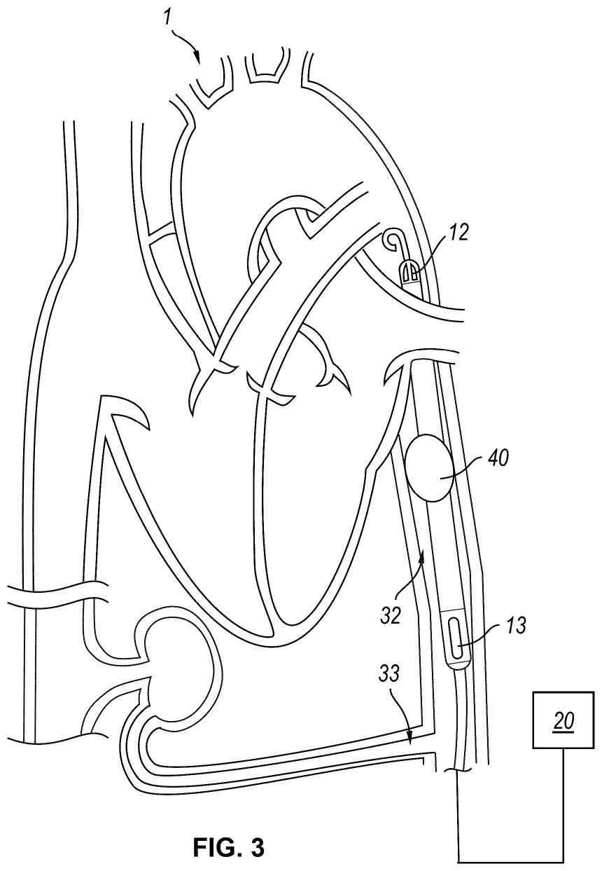

A second configuration (see FIG. 2) of a Pusher therapy may include a microaxial flow pump, such as a percutaneous blood pump, with an inlet (12) positioned in the descending aorta (32) of the patient and the outlet (13) positioned near to, and in some embodiments just superior to the renal artery (33). In some embodiments, such a configuration may have the potential benefit of having the high pressure outlet positioned very near the renal artery, which may provide an enhanced ability to increase renal artery pressure. Further, in this embodiment, mean renal artery pressure (e.g., both systole and diastole) may be augmented, which is different than the diastolic only augmentation observed with a transvalvular configuration or location (see, e.g., FIG. 1 and FIG. 3). As will be appreciated, this location however may not directly unload the LV and, thus, may only provide modest LV afterload reduction. As with the Pusher therapy of FIG. 1, the pump in this second configuration may also be operably connected to a controller (20), which may be configured to increase and/or decrease the level of support provided by the pump to achieve the desired therapy.

A third configuration of a Pusher therapy is shown in FIG. 3 and may include a microaxial flow pump, such as a percutaneous blood pump, with an inlet (12) positioned in the descending aorta (32) of the patient and an outlet (13) positioned near to, and in some embodiments just superior to the renal artery (33) of the patient. Such a configuration may augment diastole.

In some embodiments, an artificial valve structure (40) may be used to separate the inlet and outlet of the pump to reduce potential recirculation and improve the pumps hydrodynamic efficiency, which may increase the pressure increase in the renal artery. As will be appreciated, any suitable valve structure may be used in these embodiments. As will be further appreciated, other suitable structures may be used to separate the inlet and outlet of the pump, such as an inflatable balloon or a self-expanding covered stent like structure. In some embodiments, this third configuration may force all aortic flow through the pump and therefore rely on the pump to provide perfusion to all organs inferior to the inlet. As also shown in FIG. 3, the pump may be operably connected to a controller for controlling the Pusher therapy provided to the patient.

Although an artificial valve structure is shown in this view, it will be appreciated that in some configuration, such as that shown in FIG. 1 where the pump may be placed across the aortic valve, the valve of the heart (referred to herein as a natural valve) may act to separate the inlet and outlet of the pump, which may also affect the diastolic pressure augmentation of the kidney. In such embodiments, an artificial valve structure need not be used with the pump.

As will be appreciated, the kidney autoregulates its function in response to, among other things, changes in pressure and flow. In some embodiments, this autoregulation may be reduced or minimized by timing pressure and flow augmentation to the certain points in the cardiac cycle. In some embodiments, if a Pusher therapy is used and is a pump, may mean timing the support (e.g., RPMs) to the cardiac cycle. If the pusher is a flow enhancer/obstructer (e.g., a wing or nozzle) it may be advantageous to actuate the flow enhancer timed to the cardiac cycle. In some embodiments, pressure and flow may be augmented during diastole, which may reduce renal autoregulation. Furthermore, in some embodiments, this autoregulation may be reduced or minimized by varying the renal pressure augmentation profile from just diastolic pressure augmentation (see, e.g., FIG. 1 and FIG. 3) to mean renal pressure augmentation (see FIG. 2). Other autoregulation also may be utilized in other embodiments, as disclosed herein.

In some embodiments, a combination of the pump location (e.g., positioned closer to the kidney) and inclusion of a valve (e.g., natural or artificial) may increase diastolic pressure augmentation and pressure gradient. For example, in some embodiments, further from the renal artery, the diastolic pressure may increase by more than 7 mm Hg, such as between 7 and 10 mmHg. In other embodiments, closer to the renal artery, the diastolic function may increase by more than 20 mmHg, such as between 20 and 30 mm Hg because of the location and/or inclusion of the valve.

### Puller Therapies

As described herein, various anatomical locations and configurations of the Puller therapy may be deployed in the patient.

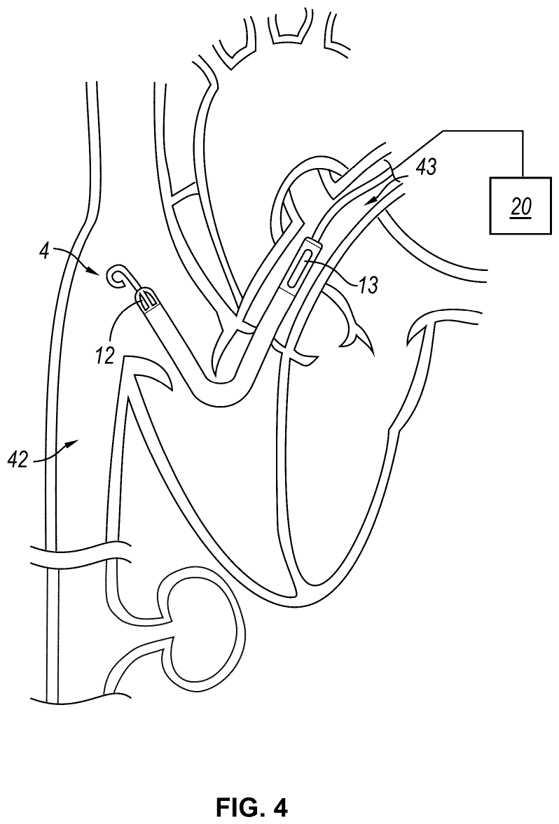

A first configuration of the Puller therapy, shown in FIG. 4, may include a microaxial flow pump, such as a percutaneous blood pump, with an inlet (12) positioned in the inferior vena cava, such as the superior portion of the inferior vena cava (IVC) (42) or right atrium (RA) (4) and an outlet (13) positioned in the pulmonary artery (43). In some instances, the inlet in the superior IVC/RA may have the potential of decreasing renal vein pressure. This configuration also may have the advantage of bypassing the right ventricle (RV), thereby decoupling renal vein pressure from RV function.

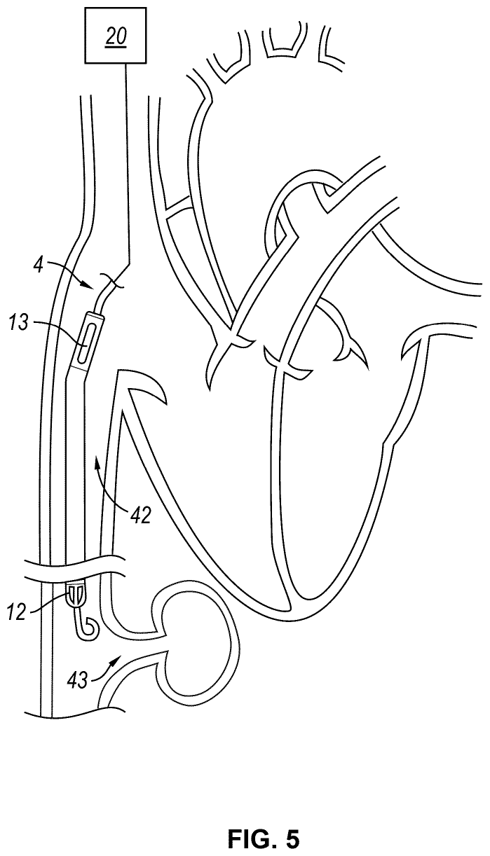

A second configuration of a Puller therapy, which can be seen in FIG. 5, may include a microaxial flow pump, such as a percutaneous blood pump, with an inlet (12) positioned in the IVC (42), such as just superior to the renal vein (43), and an outlet (13) positioned in the superior IVC (42) or RA (4). In some embodiments, such a configuration may confer the advantage of locating the low-pressure pump inlet near the renal vein with an increased ability to decrease renal vein pressure (e.g., at a fixed mechanical work). In some embodiments, this configuration may rely on the blood deposited into the RA to be pumped out by the native RV. As will be appreciated, in some patients, such as those having RV failure, the native RV may not be able to pump a sufficient volume of blood. Accordingly, in some embodiments, for such patients, other configurations may be needed to achieve a desired Puller therapy.

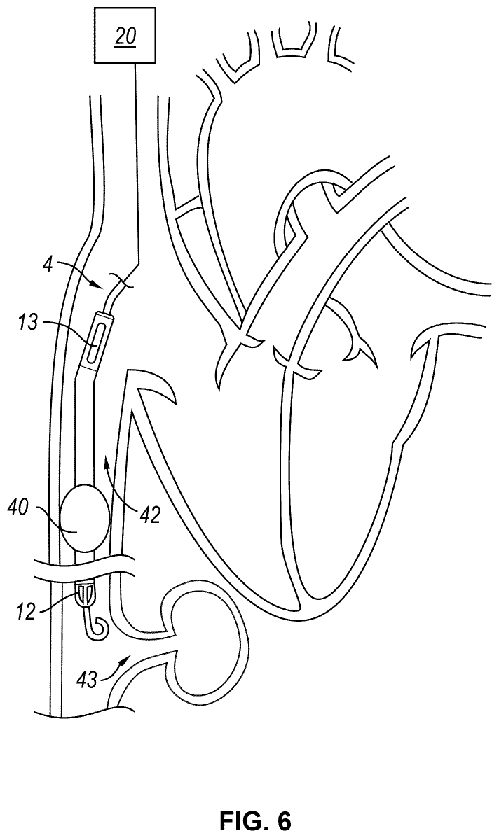

FIG. 6 shows a third configuration of Puller therapy, which may include a microaxial flow pump, such as a percutaneous blood pump, with an inlet (12) positioned in the IVC (42), such as just superior to the renal vein (43), and an outlet (13) positioned in the superior IVC (42) or RA (4).

In some embodiments, an artificial valve structure (40) may be used to separate the inlet and outlet of the pump, such as to reduce potential recirculation and improve the pumps hydrodynamic efficiency. In some embodiments, this may increase the pressure drop in the renal vein, pressure gain in the RA and the overall increase in flow. As will be appreciated, any suitable valve structure may be used in such embodiments. As will be further appreciated, other suitable structures, such as an inflatable balloon, or a self-expanding covered stent structure may be used to separate the inlet and outlet and to reduce potential recirculation. In some embodiments, the third configuration may force all IVC flow through the pump. Accordingly, the third configuration may rely on the on the pump to return all lower extremity venous return to the heart.

Again, although an artificial valve is shown for separating the inlet and outlet, in other embodiments, a valve of the heart (see, e.g., FIG. 4), referred to herein as a natural valve, may separate the inlet and outlet of the pump. In such embodiments, the pump need not include an artificial valve structure.

In some embodiments, the Pusher and/or Puller therapies may be tailored to achieve a desired transrenal gradient, to increase renal perfusion, spare renal injury, and improve renal function and, in turn, patient outcomes. In some embodiments, therapy feedback in the form of renal function (e.g., urine output, natriuresis, serum creatine, GFR, BUN/creatine ratio), volume status changes (e.g., urine output, edema, congestion) biomarkers for HF status (natriuretic peptides etc.), and hemodynamics may be used to tailor the pusher and puller therapy.

In addition to feedback from physiologic markers, in the case where both a pusher and puller therapies are used (see below) the pusher and puller devices may be in communication with one another to titrate a therapy. In some embodiments, such communication may be achieved by operably connecting the pusher and puller therapies to a single controller, which may be used to control the level of support. In some embodiments, the communications may help optimize intraventricular coupling or help optimize transrenal pressure gradients, increase renal perfusion, spare renal injury, and improve renal function. For example, if arterial blood pressure into the renal artery improves (cither via the pusher therapy or natural physiology) the venous puller may not need to decrease venous pressure as much to achieve the same trans renal gradient and therefore the puller pump effects could be down titrated (RPM decrease). In another example, if the patient is decongesting and losing volume and therefore CVP/RAP may be decreasing due to loss of volume, the arterial pusher pump may be down titrated (e.g., RPM decrease) and still maintain the same target transrenal gradient, filtering and/or renal perfusion status.

As will be appreciated, the kidneys play an important role in cardiovascular physiology because of their ability to control volume status and therefore cardiac preload and afterload.

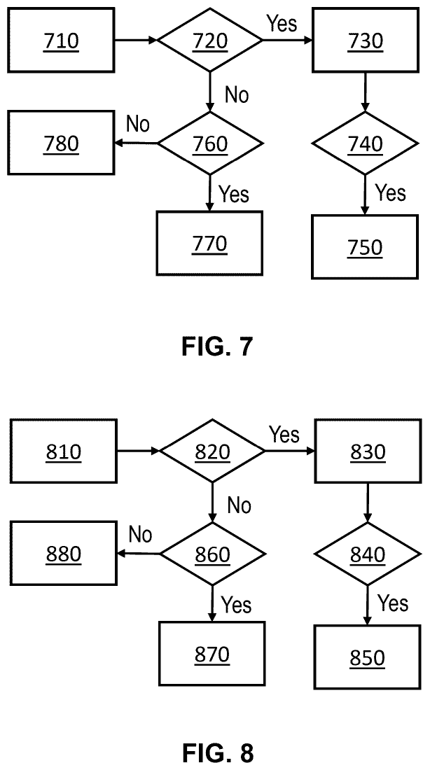

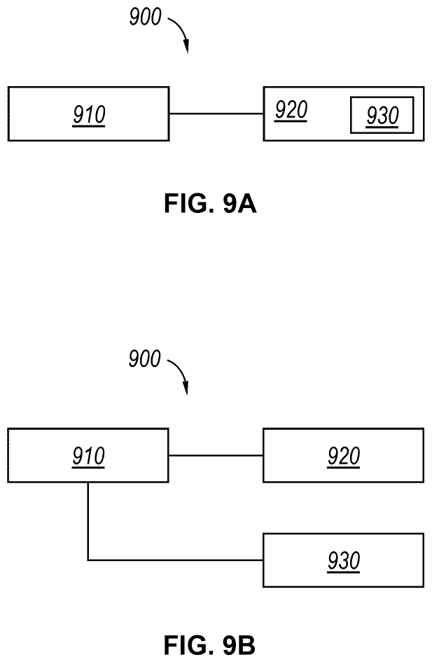

However, as will also be appreciated, other organs can often be damaged and may function poorly from hypoperfusion (decreased arterial blood flow) or congestion (increased venous pressure). Accordingly, the inventors have recognized that these other organs may benefit from pusher and puller therapies, respectively. For example, skeletal muscles, the intestines and stomach, and the liver are organs that may benefit from improved perfusion (pusher) and relieved venous congestion (puller). See, for example, FIGS. 7 and 8.

In FIG. 7, a method is shown for responding to situations where a patient may be initially determined (710) to have signs and/or symptoms of congestion. It may then be determined (720) if the patient has hypoperfusion, for example, hypoperfusion may be present if the patient exhibits a systolic blood pressure (SBP)<90 mmHg or a cardiac index (CI)<2.2 L/min/m², or cold extremities or elevated lactate, etc. If so, then the method may include considering an introducing pusher therapy (730) to improve renal perfusion. Further, it may also be determined (740) if there is any venous congestion. For example, venous congestion may be present if the central venous pressure (CVP) and/or right atrial pressure (RAP)>10 mmHg, if there jugular vein distention, or peripheral edema, or high intraabdominal pressure, etc. If so, the method may include considering adding (750) puller therapy to the pusher therapy to relieve the venous congestion.

If, however, there was no hypoperfusion, it may also be determined (760) if there is any venous congestion, using a similar approach to that in step 740. If so, the method may include considering introducing (770) puller therapy to relieve the venous congestion. If not, the method may include considering (780) other sources of symptoms.

In FIG. 8, a method is shown for responding to situations where a patient may initially be determined (810) to have markers of end organ dysfunction (e.g., via liver enzymes, lactate, lactulose/mannitol, and/or renal injury markers not in a normal range). It may then be determined (820) if the patient has hypoperfusion, for example, hypoperfusion may exist if the patient exhibits a systolic blood pressure (SBP)<90 mmHg or a cardiac index (CI)<2.2 L/min/m², or cold extremities or elevated lactate, etc. If so, the method may include considering introducing pusher therapy (830) to improve renal perfusion. Further, it may also be determined (840) if there is any venous congestion, using a similar approach to that in step 740. If so, the method may include considering adding (850) puller therapy to the pusher therapy to relieve the venous congestion.

If, however, there was no hypoperfusion, it may also be determined (860) if there is any venous congestion, the method may include using a similar approach to that in step 740. If so, the method may include considering introducing (870) puller therapy to relieve the venous congestion. If not, consider (880) other sources of symptoms.

### Systems

In various aspects, a system for improving renal function may be provided. The system may include a first flow enhancer configured to increase a renal artery pressure. The system may include a second flow enhancer or flow restrictor configured to reduce a renal vein pressure.

Each flow enhancer or flow restrictor may be configured to increase a transrenal pressure gradient, increase renal perfusion, spare renal injury, and improve renal function.

In some embodiments, the first flow modifier may be a passive, non-powered, wing or nozzle. The first flow modifier also may be a microaxial pump, such as a percutaneous blood pump, such as those disclosed herein. In some embodiments, the microaxial pump may have an inlet positioned in a superior portion of an inferior vena cava (IVC) or right atrium (RA) of a patient, and an outlet positioned in a pulmonary artery. The microaxial pump also may have an inlet positioned in an inferior vena cava (IVC), such as superior to a renal vein, and an outlet positioned in the inferior vena cava (IVC) or right atrium (RA). An artificial valve (such as a balloon or stent valve) may be disposed between the inlet and the outlet. A natural valve also may be used to separate the inlet and outlet, such as when the pump may be placed across a valve in the heart, as described herein.

The first flow modifier may be operably coupled to a controller. The controller may be configured to receive information from a sensor. The controller may be configured to, based on the received information, determine when a heart is in diastole. The controller may also be configured to increase flow generated by the first flow modifier during diastole.

The second flow modifier may be a passive, non-powered, wing or nozzle. The second flow modifier also may be a microaxial pump. The microaxial pump may have an inlet in a left ventricle (LV) and an outlet in an aorta. The microaxial pump may have an inlet in a descending aorta and an outlet superior to a renal artery. An artificial valve (such as a balloon or stent valve) may be disposed between the inlet and the outlet. A natural valve also may be used to separate the inlet and outlet, such as when the pump may be placed across a valve in the heart, as described herein. The flow restrictor may be a balloon in some embodiments.

### Methods

In various aspects, a method for improving a renal function may be provided. The method may include increasing a transrenal pressure gradient by increasing a renal artery pressure, decreasing a renal vein pressure, or a combination thereof to increase renal perfusion, spare renal injury, and improve renal function.

The method may include providing a first flow modifier, as disclosed herein, configured to reduce a renal vein pressure. The method may include inserting the first flow modifier (such as a flow enhancer) such that an inlet may be disposed in a superior portion of an inferior vena cava (IVC) or right atrium (RA), and an outlet may be disposed in a pulmonary artery. The method may include inserting the first flow enhancer such that an inlet may be disposed in an inferior vena cava (IVC) superior to a renal vein, and an outlet may be disposed in the inferior vena cava (IVC) or right atrium (RA).

The method may include providing a second flow modifier (such as a flow enhancer, flow restrictor), as disclosed herein, configured to increase a renal artery pressure. The method may include inserting the microaxial pump such that an inlet may be disposed in a left ventricle (LV) and an outlet may be disposed in an aorta. The method may include inserting the microaxial pump such that an inlet may be disposed in a descending aorta and an outlet may be disposed superior to a renal artery.

The method may include receiving information related to when a heart is in diastole. The method may include increasing flow generated by the first flow enhancer during diastole.

The method may include determining an adjustment to a renal artery pressure or a renal vein pressure based on information received from one or more sensors. The information from the one or more sensors may be related to renal function, volume status changes, a biomarker for heart failure status, or hemodynamics. The information related to renal function may include information related to urine output, serum creatine, GFR, BUN/creatine ratio, natriuresis (e.g., serum and or urine sodium) or a combination thereof. The information related to renal injury may include neutrophil gelatinase-associated lipocalin (NGAL), or Cystatin C, TIMP-2, NAG, IGFBP-7, KIM 1, or a marker of renal oxygenation. The information related to volume status changes may include information related to urine output, edema, signs or symptoms of congestion, or a combination thereof. The biomarker for heart failure status may be a natriuretic peptide.

The method may include determining an adjustment to the first flow enhancer based on information from a sensor on the second flow enhancer or flow restrictor. The method may include determining an adjustment to the second flow enhancer or flow restrictor based on information from a sensor on the first flow enhancer.

The method may include, after achieving a desired transrenal pressure gradient, controlling the transrenal pressure gradient by adjusting a renal artery pressure, a renal vein pressure, or a combination thereof to increase renal perfusion, spare renal injury, and improve renal function.

In various aspects, a method may be provided for determining treatment of a patient with signs and symptoms of congestion, or with markers of end organ function not in a normal range. The method may include recommending a first flow enhancer configured to increase a renal artery pressure if the patient is experiencing hypoperfusion. The method may include recommending a second flow enhancer or flow restrictor configured to reduce a renal vein pressure if the patient is experiencing venous congestion.

In some embodiments, the method may include determining a patient is experiencing hypoperfusion if a systolic blood pressure <90 mmHg, a cardiac index <2.2 L/min/m², the patient has cold extremities, the patient has elevated lactate, or other indicators of renal hypoperfusion such as renal injury markers (e.g. Neutrophil gelatinase-associated lipocalin (NGAL) or kidney injury molecule 1 (KIM 1) or tissue inhibitor of metalloproteinase (TIMP)). The method may include determining a patient is experiencing venous congestion if central venous pressure (CVP) or right atrial pressure (RAP)>10 mmHg, the patient has jugular vein distention, high intraabdominal pressure, or the patient has peripheral edema.

The method also may include providing a first flow enhancer, as disclosed herein, configured to increase a renal artery pressure. The method may include inserting the first flow enhancer such that an inlet may disposed in a superior portion of an inferior vena cava (IVC) or right atrium (RA), and an outlet may be disposed in a pulmonary artery. The method may include inserting the first flow enhancer such that an inlet may be disposed in an inferior vena cava (IVC) superior to a renal vein, and an outlet may be disposed in the inferior vena cava (IVC) or right atrium (RA).

The method may also include providing a second flow enhancer or flow restrictor, as disclosed herein, configured to reduce a renal vein pressure. The method may include inserting the microaxial pump such that an inlet may be disposed in a left ventricle (LV) and an outlet may be disposed in an aorta. The method may include inserting the microaxial pump such that an inlet may be disposed in a descending aorta and an outlet may be disposed superior to a renal artery.

As described herein, a method of improving mean or diastolic renal pressure is disclosed (such as by using one or more of the above-described systems). In some embodiments, the method may include timing the pump RPM to the cardiac cycle to differentially argument diastole versus systole. In other embodiments, the method may include providing a valve, either a natural valve or a synthetic valve (e.g., a balloon or covered stent-like structure) and deploying a microaxial flow pump across the valve. In such embodiments, the method may include intrinsically increasing diastolic pressure over systolic pressure, irrespective of RPM timing. As will be appreciated, in embodiments in which the valve is removed, the method may include augmenting both the systole and diastole or mean pressure (e.g., via timing the pump).

As described herein, pusher and/or puller therapies may be used for addressing heart failure (see, e.g., FIGS. 7 and 8). Pusher and/or puller technologies also may be used for other acute and/or chronic illnesses, such as for acute kidney injury (AKI) experienced during a percutaneous coronary intervention (PCI), during cardiac surgery, cardiogenic shock, and/or delayed graft function in kidney transplants. As with the methods described herein, such a therapy (and method) may first consider whether there is some hypoperfusion experienced by the patient that may suggested considering a pusher therapy. If such an instance is experienced, a pusher therapy may be employed to improve renal perfusion. In such instances, if venous congestion is also detected, a puller therapy may be added in addition to the pusher therapy to relieve such congestion. If, hypoperfusion is not experienced, but instead only venous congestion is seen in these acute and/or chronic instances, then only a puller therapy may be employed to relieve the congestion.

In some embodiments, a system may be developed (e.g., by employing an algorithm, stored one or more non-transitory computer readable storage devices and collectively executed by one or more processing units coupled to the storage device(s)) to predict when a pusher and/or puller therapy may be needed for a given patient. In such embodiments, as will be appreciated in view of the above method, the algorithm may take one or more patient parameters indicative of congestion and/or hypoperfusion, for example, to predict whether a pusher and/or puller therapy may be beneficial. In some instances, the algorithm also may determine first whether patient profiles may correlate with each type of support. In some embodiments, the system may allow a clinician to enter in different patient parameters to determine at one threshold parameters a patient may need a pusher and/or puller therapy. In some embodiments, the system may interface with electronic health records (EHRS) and may employ various techniques to look for indicators of renal deterioration which may suggest the need for a pusher and/or puller technology.

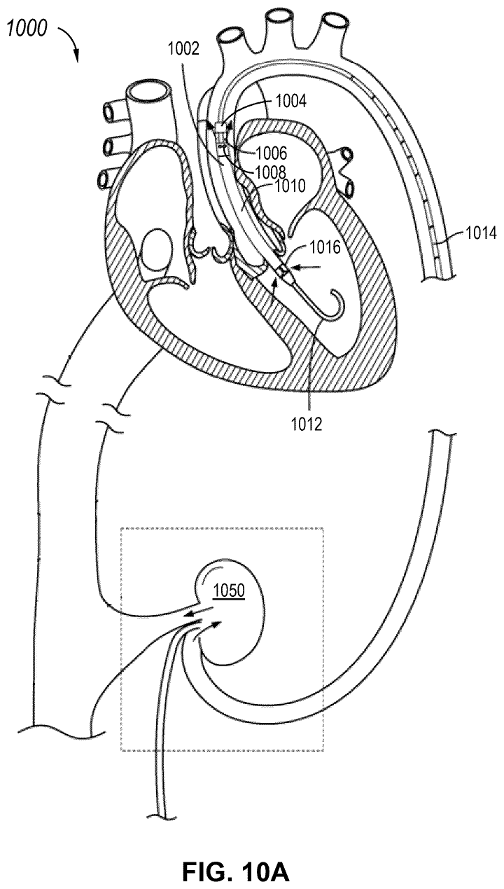

In some embodiments, a system for cardiorenal syndrome (CRS) therapy may be provided. Referring to FIG. 9A, a system (900) may include a therapy-delivery device (920) configured to be disposed within a patient. The therapy-delivery device may be a mechanical circulatory support device, such as a blood pump. The therapy-delivery device also may be a blood flow or pressure restrictor. In other embodiments, the therapy-delivery device may be a blood flow or pressure enhancer.

The therapy-delivery device may be a blood pump disposed in a blood vessel forming part of a patient's arterial circulation. The therapy-delivery device may be a blood pump disposed in a blood vessel forming part of a patient's venous circulation. The therapy-delivery device may be a blood flow or pressure restrictor disposed in a blood vessel forming part of a patient's arterial circulation. The therapy-delivery device may be a blood flow or pressure restrictor disposed in a blood vessel forming part of a patient's venous circulation. The therapy-delivery device may be a blood flow or pressure enhancer disposed in a blood vessel forming part of a patient's arterial circulation. The therapy-delivery device may be a blood flow or pressure enhancer disposed in a blood vessel forming part of a patient's venous circulation. In some embodiments, the therapy-delivery device may include a combination of these disclosed devices. In some embodiments, only a single therapy-delivery device may be provided. In some embodiments, a plurality of therapy-delivery devices may be provided.

In some embodiments, when a plurality of devices is provided, the devices may be disposed at same part of the patient's circulation (e.g., in arterial or venous circulation). For example, a blood pump and an inflatable balloon blood flow restriction device may both be located in a patient's venous circulation.

In some embodiments, when a plurality of devices is provided, the devices may be disposed at different locations. For example, in some embodiments, one device may be disposed in a patient's arterial circulation, and one device may be disposed in a patient's venous circulation.

The system may include a controller (910) operably coupled to the therapy-delivery device (920) (or to a plurality of devices). The controller may be configured to adjust a therapy delivered by the therapy-delivery device based on a parameter related to renal function. As described herein, the parameter related to renal function may be sensed in various ways. As shown in FIG. 9A, in some embodiments, a diagnostic sensor (930) may be integrated into the therapy-delivery device, where the diagnostic sensor is configured to provide the parameter. In some embodiments, the diagnostic sensor is a real-time diagnostic sensor. The diagnostic sensor also may be configured to measure the parameter at prescribed times (e.g., every 5 second, 10 seconds, 1 minute, 2 minutes, 5 minutes, 10 minutes, or longer), such as to provide feedback contemporaneous with a patient therapy.

Referring to FIG. 9B, the diagnostic sensor (930) may also be located remotely from the therapy-delivery device (920). For example, the diagnostic sensor may be disposed on an implantable device, such as a separate implantable medical device. The separate implantable medical device may be a second therapy-delivery device in some embodiments. The sensor or detector may be located extracorporeally. For example, the sensor or detector may be disposed on a wearable device. The sensor or detector also may be disposed remotely from the patient. For example, in some embodiments, the sensor or detector may be disposed on a cart holding the controller.

In some embodiments, the diagnostic sensor may be configured to detect an optical change in response to a measured biomarker. For example, the sensor may be configured to either directly or indirectly provide a response to the presence of a fluorescent taggant coupled to a biomarker that had previously been introduced to the bloodstream, that the kidney may then filter out over time. By understanding the rate of filtering, a condition of the kidney can be assessed.

The real-time diagnostic sensor may be configured to detect a change in electrical properties in response to a measured biomarker. For example, the sensor may be configured to detect a change in conductivity or resistance of the blood as the presence of the biomarker changes. The real-time diagnostic sensor may be configured to detect a mechanical change (e.g., a pressure, a flow rate, a force, etc.) responsive to a measured biomarker.

The parameter related to renal function may be a hemodynamic parameter, a renal function parameter, or a combination thereof. For example, a governing equation of direct glomerular filtration rate (GFR) may be GFR=RBF x filtration fraction, where RBF is a renal blood flow. It will be understood that data on its components may therefore help to guide therapy. The hemodynamic parameter may be renal blood flow, cardiac index, or a combination thereof. The renal function parameter may be estimated glomerular filtration rate (eGFR), which may be measured by, e.g., serum creatine. The renal function parameter may be direct glomerular filtration rate (GFR). The renal function parameter may be blood urea nitrogen (BUN). The renal function parameter maybe serum creatinine. The renal function parameter may be urinary output. The renal function parameter may be urinary sodium concentration. The renal function parameter may be total urinary sodium content. The renal function parameter maybe fractional sodium excretion (FeNa). The renal function parameter may be a renal injury marker. The renal injury marker may be Neutrophil gelatinase-associated lipocalin (NGAL), NAG (N-acetyl-beta-D-glucosaminidase), Cystatin C, TIMP-2 (Tissue Inhibitor of Metalloproteinase-2), IGFBP-7 (Insulin-like Growth Factor-Binding Protein 7), kidney injury molecule 1 (KIM 1), or a marker of renal oxygenation. The renal function parameter may be a combination of eGFR, GFR, BUN, SCr, urinary output, urinary sodium concentration, total urinary sodium content, and/or a renal injury marker.

There may be one or more thresholds for each renal function parameter to help guide therapy. For example, as is known, considering the NGAL marker, a range of <50 ng/ml indicates a low risk of acute kidney injury (AKI), a range of 50-149 ng/ml indicates an equivocal risk of AKI, a range of 150-300 ng/mL indicates a moderate risk of AKI, and a range of >300 ng/ml indicates a high risk of AKI. Similarly, the normal range of Cystatin C is around 0.62-1.15 mg/L, so values outside that range may indicate particular therapies are needed. Further, as is known, according to the optimal prediction of AKI, a [TIMP-2]·[IGFBP7] value of >0.3 (ng/ml)²/1000) may be considered positive and a value of ≤0.3 (ng/ml)²/1000 was considered negative. Alternatively, or in addition, in urine, KIM-1 concentration has been shown to increase up to 3-7 ng/ml from its normal concentration of less than 1 ng/ml; KIM-1 levels may begin to increase as early as, e.g., 6 hours after an ischemic insult and remained elevated for a period of 48 hours post-injury.

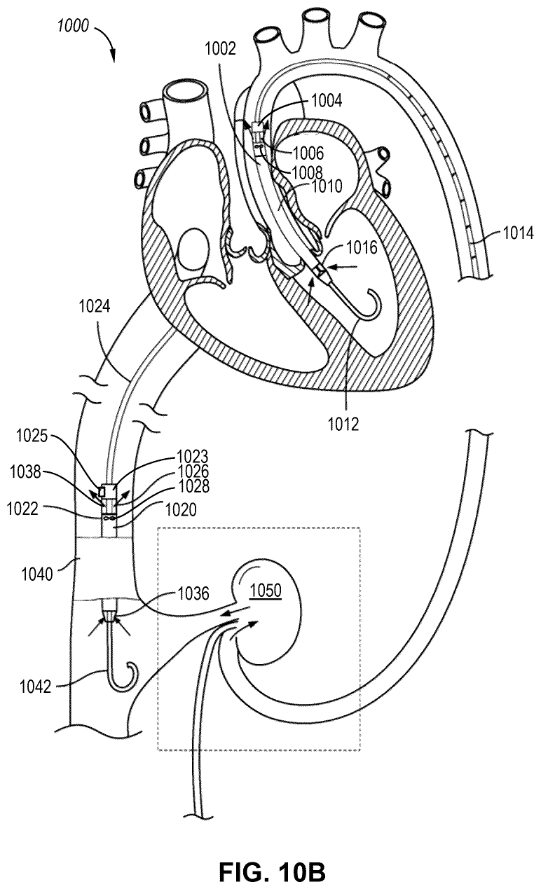

As an example, referring to FIGS. 10A-10C, embodiments of system (1000) as shown. In some embodiments, as shown in FIG. 10A, the system may include a mechanical circulatory support device, such as pump (1002) disposed in the patient's left ventricle. In some embodiments, as shown in FIG. 10B, the system may include first and second support devices, such as pump (1002) and pump (1022), disposed in the patient's inferior vena cava. In one such example, pump (1002) and pump (1022) may be IMPELLA® pumps from Abiomed, Inc. In other embodiments, the support devices may include other mechanical circulatory assist devices, such as an expandable pump, intra-aortic balloon pump or an extracorporeal membrane oxygenation system (ECMO).

In still other embodiments, such as those seen in FIG. 10C, the system may include only pump (1022), which may be disposed in the inferior vena cava.

In some embodiments pump (1002) and/or pump (1022) may control input and output into an organ, e.g., the heart and/or the kidney (1050) (see FIGS. 10A and 10B). As will be appreciated, the kidney (1050) may be the left kidney or the right kidney. In one example, pump (1002) and/or pump (1022) may control input and output into both kidneys. For example, operating one or both blood pumps may change a flow quantity and flow rate of blood through the organ, e.g., kidney. For example, operating one or both blood pumps may increase a flow quantity and flow rate of blood through the kidney.

As shown in FIG. 10A for example, pump (1002) may be a first blood pump with motor (1004) and rotor (1006) in housing (1008), cannula (1010), distal extension (1012), and catheter (1014). As shown in the example of FIG. 10A, pump (1002) may be placed with distal extension (1012) in the left ventricle, and rotor (1006) and housing (1008) in the aorta. When operating, pump (1002) may draw blood through the inlet (1016), through cannula (1010) and out through housing (1008) (sometimes referred to as the rotor shroud), unloading the heart. This, in turn, may impact the functionality of the kidneys, as disclosed herein. In some embodiments, the distal extension (1012) may act to stabilize pump (1002) in the ventricle. For example, distal extension (1012) may be a pigtail or j-shape. When operating, pump (1002) may unload the left ventricle and increases pressure in the aorta, thereby increasing arterial pressure downstream (e.g., in the kidneys). Pump (1002) may operate at a range of speeds resulting in a range of flow rates and associated increases in aortic pressure. For example, the pump (1002) may be operated at a flow rate between about 1.5 L/min and 6 L/min. In one example, the pump (1002) may be operated at a flow rate of about 5 L/min. Pump (1002) may be percutaneously or surgically inserted into a patient via the femoral artery, or via the axillary artery or via the carotid artery.

As shown in FIG. 10B, pump (1022) may be a second blood pump with motor (1023) and rotor (1028) in housing (1026), cannula (1020), distal extension (1042), and catheter (1024). As shown in the example of FIG. 10B, pump (1022) may be placed in the inferior vena cava. When operating, pump (1022) may draw blood through the inlet (1036), through cannula (1020) and out through housing (1026). Housing (1026) with rotor (1028) may be in the inferior vena cava, downstream of inlet (1036), also in the inferior vena cava. As will be understood by those of skill in the art, FIGS. 10A and 10B do not show all variations of the devices. For example, in some embodiments, the housing (1026) with rotor (1028) in pump (1022) may be upstream of the cannula (1020), such that blood is pushed through the cannula, exiting at the outlet (1038). A similar design may be used for pump (1002).

In one example, e.g., as shown in FIG. 10B, pump (1022) may include distal extension (1042) which may stabilize pump (1022) in the inferior vena cava, or at a junction between the inferior vena cava and the renal vein. For example, distal extension (1042) may be a pigtail or j-shape. Pump (1022) may also include anchoring mechanism (1040), positioned on the cannula between inlet (1036) and housing (1026) through which blood exits the pump. Anchoring mechanism (1040) may both anchor the pump (1022) at a desired position along the inferior vena cava and partially occlude the inferior vena cava to allow operation of pump (1022) across the anchoring mechanism.

In some embodiments, the anchoring mechanism may act as a valve to separate the inlet and outlet, such as to reduce recirculation and improve pump hydrodynamic performance characteristics. For example, anchoring mechanism (1040) may anchor pump (1022) between about 1-5 centimeters downstream of the renal vein. In another example, anchoring mechanism (1040) may anchors pump (1022) between about 2-3 centimeters downstream of the renal vein. In one example, anchoring mechanism (1040) may be a balloon, which can be selectively inflated to at least partially occlude the inferior vena cava. Again, in this example, the anchoring mechanism may act to separate the inlet and outlet as a valve. For example, the size, shape, material, and position of the balloon on the cannula (1020) may be selected to achieve different levels of occlusion in the inferior vena cava.

In another example, anchoring mechanism (1040) may be an expandable cage. For example, anchoring mechanism (1040) may be a self-expanding cage (e.g., Nitinol) which may be surrounded by a sheath for insertion and self-expands once the sheath is removed in situ. The cage may brace up against walls of the inferior vena cava and secure pump (1022) in position. In one example, the cage may taper proximally and distally along the cannula, and be covered by a biocompatible cover material, to partially occlude the inferior vena cava.

Partial occlusion of the inferior vena cava, in combination with operation of pump (1022), may draw blood from a location within the inferior vena cava and/or renal vein, to a location downstream of the pump inlet (1036), may result in a pressure drop. The pressure drop may be measured as a pressure drop in the inferior vena cava upstream of pump (1022) (e.g., proximate the renal vein), or a pressure drop in the renal vein. Alternatively, the pressure drop can be measured as a drop across the kidney between arterial pressure going into the kidney, and venous pressure coming out of the kidney (e.g., in the renal vein or transrenal pressure gradient). Indeed, in some aspects, the transrenal pressure gradient may be a parameter that is measured and used to guide therapy of the patient.

As with the first pump, pump (1022) may also be percutaneously or surgically inserted into a patient, such as via the femoral vein or via the jugular vein or via the subclavian vein. In one example, pump (1022) and pump (1002) may be inserted through different percutaneous access points. Alternatively, pump (1022) and pump (1002) may be inserted through a same percutaneous access point (e.g., subclavian vein).

In one example, each pump (e.g., pump (1002), pump (1022)) may include one or more sensors, such as, e.g., a pressure sensor or a diagnostic sensor (e.g., a real time diagnostic sensor). For example, both pumps may include an integrated pressure sensor, such as a differential pressure sensor, a piezoelectric pressure sensor, or an optical pressure sensor. In FIG. 10B, a real-time diagnostic sensor (1025) may be integrated into the second pump (1022). In another example, a separate sensor may be provided, such as one introduced on a sensor wire, or a Swan-Ganz catheter.

In some examples, the diagnostic sensor (e.g., the real-time diagnostic sensor) may generate an electrical and/or optical signal proportional to a detected concentration of a compound filtered out of the blood by the kidneys, and the electrical signal may be generated sent to a controller (e.g., an AUTOMATED IMPELLA CONTROLLER® (AIC) control device from Abiomed, Inc.). The signal may be displayed, or the signal may be converted (e.g., to a qualitative or quantitative value representative of the signal), and the converted values are displayed. For example, in some embodiments, multiple electrical and/or or optical signals may be gathered by the controller (e.g., multiple data points from different points in time), and a rate of change may be displayed.

Each pump (e.g., pump (1002) and pump (1022)) may be connected to a controller, e.g., an AIC, which receives data from the pump and the sensor associated with the pump (e.g., either an integrated sensor or a separate sensor), and generates for display to the user (e.g., a medical professional) information on cardiac output, and/or renal function (e.g., GFR, renal blood flow, urine output, natriuresis, etc.). As will be appreciated, in some embodiments, the pump may be connected to the same controller, although each pump may be connected to a separate controller. In such an example, each controller may be connected to an auxiliary monitor which may monitor and/or control the first and second controllers.

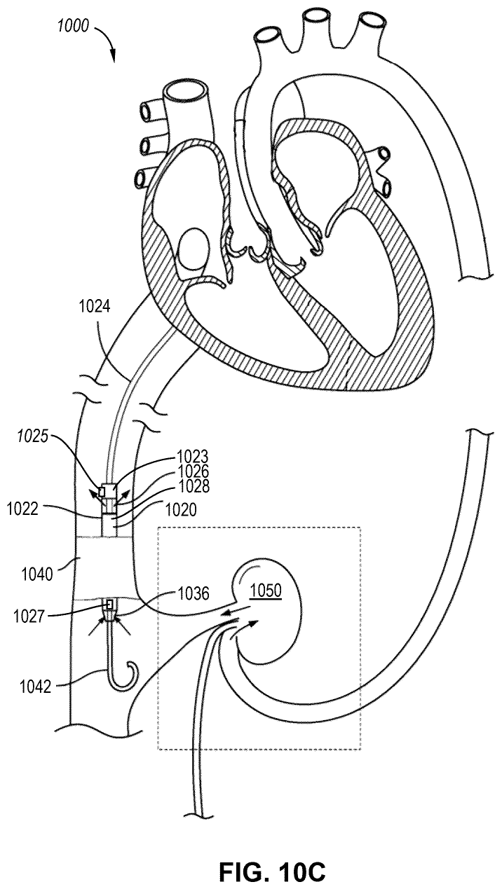

Referring to FIG. 11, the controller may be configured to receive (1110) information. This may include receiving (1112) data from a user (e.g., user-entered data). The user-entered data may include data related to renal function. As will be understood, data related to renal function may generally relate to date relevant to how the kidneys are functioning. This may include not just GFR, but urine output, natriuresis, protein urea, markers of renal dysfunction, etc.

Receiving information may include receiving (1114) data related to renal function from a diagnostic sensor, such as the real-time diagnostic sensor.

In some embodiments, the data related to renal function may include one or more parameters related to renal function, as disclosed herein. In some embodiments, controller may be configured to derive or determine (1120) the parameter(s) related to renal function based on the received information, including the data related to renal function. In some embodiments, a single parameter is determined or derived. In some embodiments, a plurality of different parameters is determined or derived.

The controller may be configured to adjust (1130) a therapy delivered by the therapy-delivery device based on a parameter related to renal function. Adjusting the therapy may include increasing or decreasing therapy dose. Increasing or decreasing the therapy dose may include increasing or decreasing a rotational speed of a blood pump. Adjusting the therapy may include increasing or decreasing an intended duration of therapy. Adjusting the therapy may include increasing or decreasing a duty cycle of therapy. In some embodiments, adjusting the therapy may include replacing the blood pump with a blood pump capable of higher flow rates. In some embodiments, a first determined parameter may be used to adjust the therapy of the first pump while a second determined parameter may be used to adjust the therapy of the second pump. The first and second determined parameters may include measurements from the same or different areas of the patient (e.g., the venous system and/or the arterial system).

In some embodiments, the determined parameters may be compared to a prescribed value of renal function. In such embodiments, when the determined parameter falls outside of a prescribed value indicative of renal function, the controller may be configured to adjust the therapy being delivered to the patient. As will be appreciated, in such embodiments, the patients may have one or two installed mechanical support devices (e.g., pump (1002) and/or pump (1022)), with a set level of support. In such embodiments, in response to the determined value, adjusting the therapy may include increasing and/or decreasing the level of support to improve renal function.

In some embodiments, the controller may be configured to determine (1140) adequacy of therapy withdrawal timing and/or duration of therapy based on the determined parameter related to renal function. For example, in some embodiments, after adjusting the therapy of the patient, the diagnostic sensor may sense and/or derive an additional parameter relating to kidney function (e.g., repeating some or all of step 1110). In response to this value, the controller may be configured to determine if additional therapy is needed (e.g., at the same or different level) or if the support should be adjusted, such as if the sensed and/or derived parameter falls outside a prescribed value of renal function (e.g., outside an optimal range). In some embodiments, if the sensed and/or derived parameter is indicative of improved renal function (e.g., when compared to the desired value or range), the controller may be configured to suggest withdrawal and/or duration of the therapy.

As will be appreciated, steps 1110-1140 may continue to be repeated, such as every time the sensor is configured to sense one or more parameters of renal function.

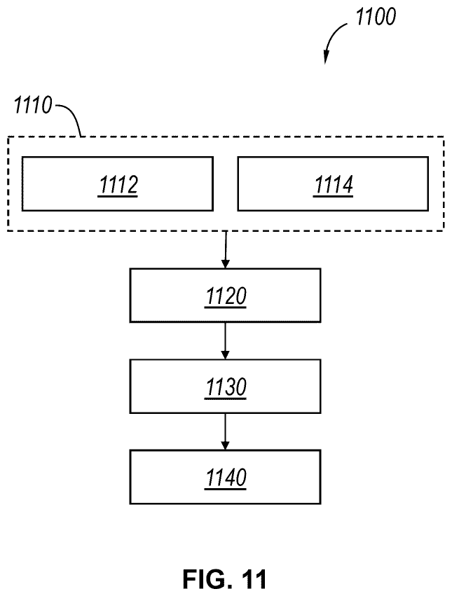

In various aspects, a method for cardiorenal syndrome (CRS) therapy may be provided. Referring to FIG. 12, the method (1200) may include disposing (1210) a therapy-delivery device within a patient. As disclosed herein, the therapy-delivery device may be mechanical circulator support device, such as a blood pump, a blood flow or pressure restrictor or enhancer, which may be disposed within a patient's arterial circulation or venous circulation. In some embodiments, the method may include inserting a single therapy-delivery device, while in other embodiments, the method may include inserting multiple therapy-delivery devices.

The method also may include receiving (1220) information. In some embodiments, this may include receiving (1222) data from a user (e.g., user-entered data). For example, the user-entered data may include data related to renal function. Receiving information also may include receiving (1224) data related to renal function from a diagnostic sensor, such as a real-time diagnostic sensor. As described herein, the sensor may be pressure sensor (e.g., an integrated pressure sensor, such as a differential pressure sensor, a piezoelectric pressure sensor, or an optical pressure sensor). In some embodiments, the sensor may be integrated into one or both of the therapy-delivery devices. In other embodiments, a separate sensor may be provided, such as one introduced on a sensor wire, or a Swan-Ganz catheter.

In some embodiments, the data related to renal function may include one or more parameters related to renal function. In some embodiments, the method may include determining or deriving (430) parameter(s) related to renal function based on the received information, including the data related to renal function. In some embodiments, a single parameter is determined or derived. In some embodiments, a plurality of different parameters is determined or derived.

The method may include adjusting (1240) a therapy delivered by the therapy-delivery device based on information related to renal function. In some embodiments, the method also may include starting therapy (1239) delivered by the therapy-delivery device. In some embodiments, adjusting the therapy may include increasing or decreasing therapy, such as that selected for the patient when the therapy was started (e.g., in step 1039). In embodiments in which therapy is not started, adjusting the therapy may include starting the therapy-delivery device. In some embodiments, increasing or decreasing the therapy may include increasing or decreasing a rotational speed of a blood pump. Adjusting the therapy may include increasing or decreasing an intended duration of therapy. Adjusting the therapy also may include increasing or decreasing a duty cycle of therapy.

The method may include determining (1250) adequacy of therapy withdrawal timing and duration based on the parameter related to renal function, as described herein. The method may include causing (1260) the therapy withdrawal timing and/or duration to be modified.

As will be appreciated, steps 1220-1260 may be repeated, such that the value of kidney function can be consistently monitored, with support adjusted accordingly.

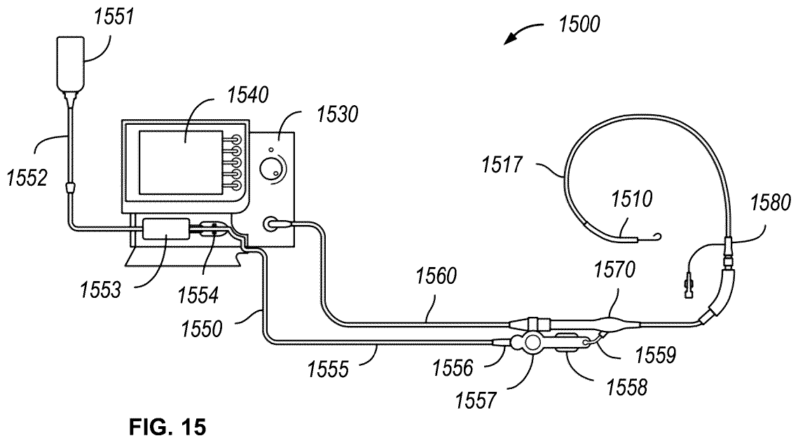

Referring to FIG. 15, a blood pump assembly (1500) may include a blood pump (1510) fluidically connected (e.g., through a tube (1552) to a container (1551) (such as a purge bag) that contains a purging fluid as disclosed herein, through a purging device (1553). The blood pump assembly (1500) also may include a controller (1530) (e.g., an AIC from Abiomed, Inc., Danvers, MA), a display (1540), a connector cable (1560), a plug (1570), and a repositioning unit (1580). As shown, the controller (1530) may include a display (1540). Controller (1530) may monitor and controls blood pump (1510). During operation, purging device (1553) may deliver a purge fluid (such as a saline-based purge fluid) to blood pump (1510) through a first line (1550, 1555) (e.g., a tube), through one or more components (1556, 1557, 1558, 1559) and through a catheter tube (1517), such as to prevent blood from entering the motor (not shown) within a motor housing of the pump. Connector cable (1560) may provide an electrical connection between blood pump (1510) and controller (1530). Plug (1570) connects catheter tube (1517), purging device (1553), and connector cable (1560). In some embodiments, plug (1570) may include a memory for storing operating parameters in case the patient needs to be transferred to another controller. Repositioning unit (1580) may be used to reposition blood pump (1510). As shown in this view, the fluid line (1550, 1555) may be separate from the connector cable (1560) having one or more electrical wires.

In another embodiment, GFR may be evaluated by measuring the value of a tracer introduced into the blood stream of a patient. For example, in some embodiments, a purge line of a percutaneous heart pump (e.g., a purge bag, such as container (1551) in FIG. 15, connected to a pump controller, such as controller (1530) in FIG. 15), may be spiked with a known concentration of a tracer (e.g., a fluorescent, autofluorescent, photoactivated, or other tracer). In some embodiments, the concentration and volume of the tracer may be provided to the controller. During use of the blood pump, the controller may be enabled to measure the provided value of the tracer to the patient. In such embodiment, the pump connected to the pump controller may include an optical sensor. In some embodiments, the optical sensor may be disposed on a portion of the pump, such as the cannula, that is blood contact (see, e.g., sensor (1027) in FIG. 10C). For example, the sensor may be disposed on a tip of a cannula of the blood pump. In such embodiments, the sensor may be tuned to recognize the wavelength of the tracer, and the sensed data may be used to calculate the amount of tracer in the blood at any given time.

Using the sensed data, a difference between the amount of tracer provided to the patient and the amount of tracer removed the body may be calculated. In such embodiments, the difference may be compared to a prescribed “normal” range of the amount of tracer in the body. As will be appreciated, this range may vary depending upon the type of tracer selected, although the range also may be constant from tracer to tracer. In some embodiments, if the calculated difference is above the prescribed range (e.g., a residual concentration of tracer in the body is too high), a therapy describe herein may be prescribed to improve renal function. As will be appreciated, in some embodiments, the difference may be tracked over time to see if a concentration of tracer remaining in the body remains the same (e.g., too high) or decreases over time to be within, or even below the desired range.

In other embodiments, the residual concentration of tracer in body may be converted into a GFR value, indicating the speed of clearance. If the GFR falls outside the prescribed normal/healthy range as reported in literature, a renal therapy, such as those disclosed herein, may be performed. In such embodiments, the GFR may be tracked over time to see if the therapy allows the GRF of the patient to increase into (or even above) the prescribed range.

### Examples

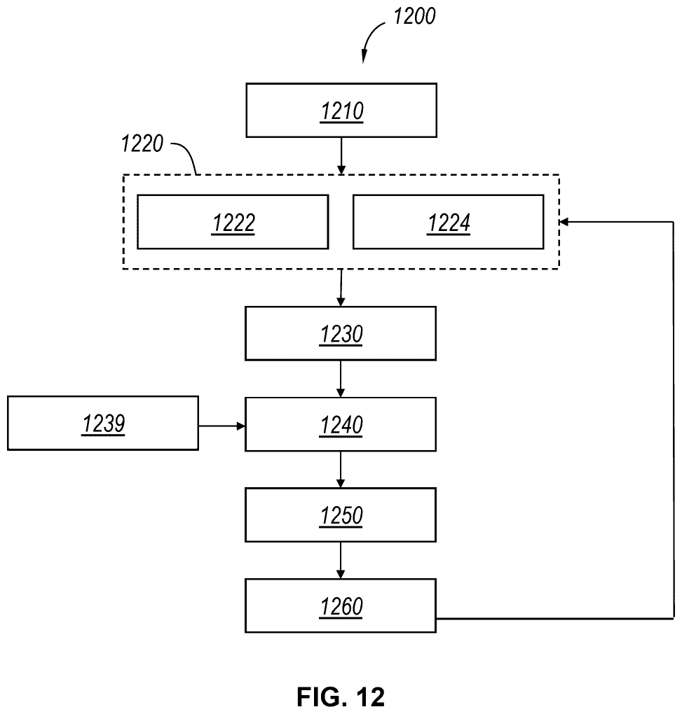

For example, referring to FIG. 13, in some embodiments, the controller may receive (1310) data while a therapy-delivery device is in operation. A parameter (such as a GFR value) may be determined (1320) based on sensor data. In some embodiments, a rate of change of the parameter may also be determined. The parameter and/or rate of change of the parameter may then be compared (1330) to a threshold.

If the parameter is outside a predefined threshold, or is declining at a rate outside a threshold rate (which may be 0), the controller may be configured to increase (1340) a pump's rotation speed until the parameter is within the threshold and/or the parameter is no longer declining.

If the parameter is increasing, or is stable, the controller may be configured to reduce (1350) pump speed. Alternatively, the controller may indicate (1360) (e.g., on a display) that the pump(s) may be removed.

As described herein, the steps may be repeated, as needed, as the patient's kidney function is monitored, and support is maintained and/or adjusted to improve renal function.

### GFR/eGFR

In some embodiments, a blood pump may be disposed in the arterial circulation to increase renal artery blood flow and pressure. In some embodiments, a blood pump may be disposed in the venous circulation to reduce renal vein pressure. In some embodiments, a first blood pump may be disposed in the arterial circulation, and a second blood pump may be disposed in the venous circulation.

The parameter used to control operation of the therapy-delivery devices may be a marker of renal function such as glomerular filtration rate (GFR). The parameter, or a rate of change of the parameter, may be determined based on sensor data.

The GFR can be estimated based on measured serum creatinine concentration corrected for patient demographics (estimated GFR, or eGFR). This method is the clinical standard.

However, eGFR it is inaccurate for some patients, is collected infrequently, and currently has a lengthy lag time from sample collection to results. In some instances, eGFR may lag actual AKI events by 24-48 hours.

The GFR can instead be directly calculated by measuring the clearance of a molecule from the plasma into the urine that is freely filtered by the kidney (e.g., inulin). This is a technically challenging method that is rarely conducted clinically, is collected infrequently, requires urine and plasma sampling, and has lag time (hours) from sample collection to results.

One measurement of GFR may be a direct measurement of GFR that is updated on a minute-by-minute basis and therefore allows for real-time adjustment of pump operation. One embodiment may use a fluorescently labeled tracer that is eliminated by the kidneys and optically detectable through the skin on a minute-by-minute basis. The change in this optical signal relates to the removal of the tracer via the kidneys and therefore GFR can be measured, and pump speed can be adjusted on a real-time basis.

### Urine Output/Diuresis

In some embodiments, a blood pump may be disposed, e.g., such as in the arterial circulation to increase renal blood flow and pressure. In some embodiments, a blood pump may be disposed, e.g., in the venous circulation to reduce renal vein pressure in order to increase diuresis. In some embodiments, a first pump may be disposed in the arterial circulation and a second pump may be disposed in the venous circulation.

The parameter used to control operation of the therapy-delivery devices may be a marker of diuresis such as urine output (UOP) or net fluid change (fluid input-UOP), or UOP relative to the amount of diuretic administered (i.e., UOP/diuretic dose or diuretic efficiency), weight change. The parameter, or a rate of change of the parameter, may be determined based on sensor data.

Urine output can be assessed by collecting urine volume over time or by using flow sensors on urine output collection tubing. Collection of urine volume over time is limited by the graduations of the collection vessel and often requires urine to be collected for 6 or more hours to collect large enough volumes to measure.

In some embodiments, UOP is measured using a flow sensor that measures flow of urine from the patient on a minute-by-minute basis and therefore allows for real-time adjustment of pump operation.

### Natriuresis

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to increase natriuresis. In some embodiments, a first pump may be disposed in the arterial circulation and a second pump may be disposed in the venous circulation.

The parameter used to control operation of the therapy-delivery devices may be a marker of natriuresis such as sodium concentration in the plasma or the urine or derivates thereof (e.g. fractional sodium excretion). The parameter, or a rate of change of the parameter, may be determined based on sensor data.

Urine and plasma sodium can be assessed by electrolyte biochemical assays conducted on the benchtop or using point of care tests. One method for utilizing plasma sodium concentration may include use of electrochemical sensors embedded on one or more of the blood contacting surfaces of the blood pumps. These electrochemical sensors could detect changes in electrical resistivity due to increasing amounts of the sodium electrolyte and thereby measuring sodium concentration on a minute-by-minute basis and therefore allows for real-time adjustment of pump operation.

### Hematocrit

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to facilitate hemoconcentration. In some embodiments, a first pump may be disposed in the arterial circulation and a second pump may be disposed in the venous circulation.

The parameter used to control operation of the therapy-delivery devices may be a marker of hemoconcentration such as hematocrit (i.e., the proportion of blood made up of blood cells). The parameter, or a rate of change of the parameter, may be determined based on sensor data.

One method of determining hematocrit may include use of optical sensors, which may be embedded on one or more of the blood contacting surfaces of the blood pumps. Due to the known effect of red blood cells on transmission/scattering of near infrared (NIR) wavelengths, a detected optical signal relating to the transmission/scattering of one or more NIR wavelengths may be used to estimate hematocrit on a minute-by-minute basis and therefore allows for real-time adjustment of pump operation.

### Decongestion

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to facilitate decongestion. In some embodiments, a first pump may be disposed in the arterial circulation and a second pump may be disposed in the venous circulation.

The parameter used to control operation of the therapy-delivery devices may be a marker of decongestion such as hemodynamic parameters (i.e., right atrial pressure, pulmonary capillary wedge pressure, pulmonary artery diastolic pressure, left ventricular end diastolic pressure), or the above parameters of UOP/diuresis, hemoconcentration, etc. The parameter, or a rate of change of the parameter, may be determined based on sensor data.

One method of determining decongestion may include use of pressure sensors, which may be embedded on one or more of the blood contacting surfaces of the blood pumps.

### Example 2

In some embodiments, a blood pump may be disposed, e.g., in the arterial circulation to increase renal blood flow and pressure. In some embodiments, a blood pump may be disposed, e.g., in the venous circulation to reduce renal vein pressure in order to provide nephroprotection from renal injury. In some embodiments, a first pump may be disposed in the arterial circulation and a second pump may be disposed in the venous circulation.

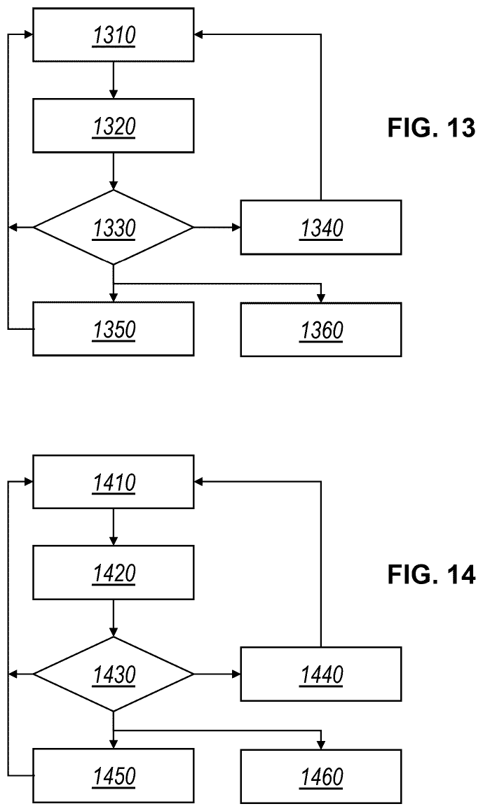

Referring to FIG. 14, in some embodiments, the controller may receive (1410) data while a therapy-delivery device is in operation. A parameter may be determined (1420) based on sensor data. In some embodiments, a rate of change of the parameter may also be determined. The parameter and/or rate of change of the parameter may then be compared (1430) to a threshold.

If the parameter is outside a predefined threshold, or is increasing at a rate outside a threshold rate (which may be 0), the controller may be configured to increase (1440) a pump's rotation speed until the parameter is within the threshold and/or the parameter is no longer increasing.

If the parameter is decreasing, or is stable, the controller may be configured to reduce (1450) pump speed. Alternatively, the controller may indicate (1460) (e.g., on a display) that the pump(s) may be removed.

### Renal Injury Biomarker

Here, the parameter used to control operation of the therapy-delivery devices may be a marker of renal injury such as NGAL, Cystatin C, TIMP-2, IGFBP-7, KIM 1, or a marker of renal oxygenation. The parameter, or a rate of change of the parameter, may be determined based on sensor data.

A preferred method of renal injury assessment could use standard laboratory values inputted into the pump control console where a predetermined algorithm adjusts pump speed. Another embodiment can embed the biochemical detection assay onto one or more of the blood contacting surfaces of the blood pumps and use optical or electrical changes related to the binding of the renal injury marker with a substrate to estimate renal injury on a minute-by-minute basis and therefore allows for real-time adjustment of pump operation.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A system for improving renal function, comprising:
a first flow modifier configured to increase a renal artery pressure; and/or
a second flow modifier configured to reduce a renal vein pressure;
wherein the system is configured to increase a transrenal pressure gradient, increase renal perfusion, spare renal injury, improve renal function, or a combination thereof.

2. The system of claim 1, wherein the first flow modifier is a microaxial pump.

3. The system of claim 2, wherein the microaxial pump has an inlet in a superior portion of an inferior vena cava (IVC) or right atrium (RA), and an outlet in a pulmonary artery.

4. The system of claim 2, wherein the microaxial pump has an inlet in an inferior vena cava (IVC) superior to a renal vein, and an outlet in the inferior vena cava (IVC) or right atrium (RA).

5. The system of claim 4, further comprising an artificial valve disposed between the inlet and the outlet.

6. The system of claim 5, wherein the artificial valve is a balloon or a covered stent valve.

7. (canceled)

8. The system of claim 1, wherein the first flow modifier is a passive, non-powered, wing or nozzle.

9. The system of claim 1, wherein the second flow modifier is a microaxial pump.

10. The system of claim 9, wherein the microaxial pump has an inlet in a left ventricle (LV) and an outlet in an aorta.

11. The system of claim 9, wherein the microaxial pump has an inlet in a descending aorta and an outlet superior to a renal artery.

12. The system of claim 11, further comprising an artificial valve disposed between the inlet and the outlet.

13. The system of claim 12, wherein the artificial valve is a balloon or a covered stent valve.

14. (canceled)

15. The system of claim 1, wherein the second flow modifier is a passive, non-powered, wing or nozzle.

16. The system of claim 1, wherein the second flow modifier is a balloon.

17. The system of claim 1, wherein the first flow modifier is operably coupled to a controller.

18. The system of claim 17, wherein the controller is configured to receive information from a sensor and, based on the information, determine when a heart is in diastole.

19. The system of claim 18, wherein the controller is configured to increase flow generated by the first flow modifier during diastole.

20. A method for improving renal function, comprising:
increasing a transrenal pressure gradient by increasing a renal artery pressure, decreasing a renal vein pressure, or a combination thereof to increase renal perfusion, spare renal injury, and improve renal function.

21-47. (canceled)

48. A method for determining treatment of a patient with signs and symptoms of congestion, or with markers of end organ function not in a normal range, the method comprising:
recommending a first flow enhancer configured to increase a renal artery pressure if the patient is experiencing hypoperfusion; and
recommending a second flow enhancer or flow restrictor configured to reduce a renal vein pressure if the patient is experiencing venous congestion.

49-67. (canceled)

68. A method for cardiorenal syndrome (CRS) therapy, comprising:
disposing a therapy-delivery device within a patient, where the therapy-delivery device is a blood pump;
receiving information related to cardiorenal function; and
adjusting a therapy delivered by the therapy-delivery device based on the information related to renal function.

69-91. (canceled)
